(19) United States
(12) Patent Application Publication
(10) Pub. No.: US 2007/0026072 A1
(43) Pub. Date: Feb. 1, 2007

(54) Benzoquinones of enhanced bioavailability

(76) Inventors: Stephen Olsen, North Bergen, NJ (US); John Alfred Doney, Washington, DC (US); Christopher Steven Shores, Marriottsville, MD (US)

Correspondence Address: International Specialty Products;Attn: William J. Davis, Esq., Legal Department, Bldg, 8, 1361 Apls Road, Wayne, NJ, 07470, US

(21) Appl. No.: 11/495,991

(22) Filed: Jul. 28, 2006

Related U.S. Application Data
(60) Provisional application No. 60/756,454, filed on Jan. 5, 2006.
(60) Provisional application No. 60/703,374, filed on Jul. 28, 2005.

(51) Int. Cl. A61K 31/12; A61K 9/14

(52) U.S. Cl. 424/486; 424/488; 514/682; 514/690

(57) ABSTRACT

Benzoquinone compositions of enhanced solubility and bioavailability are described that contain at least one benzoquinone with at least one solubility-enhancing polymer. In one embodiment, the benzoquinone is coenzyme Q1O. Described methods to produce the bioenhanced products comprise dry blending and solvent spray drying. One aspect of the method includes the steps of providing a mixture comprising benzoquinone, a solubility-enhancing polymer and a solvent and removing the solvent to form amorphous benzoquinone. Products made by the invention's compositions and methods include pharmaceuticals, nutraceuticals, cosmetic, and personal care products for man and animal.

### REFERENCE TO RELATED APPLICATIONS

This application claims the benefit of U.S. patent applications Ser. No. 60/756,454, filed Jan. 5, 2006 and 60/703,374, filed Jul. 28, 2005, the contents of which are hereby incorporated by reference.

### BACKGROUND OF THE INVENTION

The present invention is directed to compositions of bioenhanced benzoquinones and methods for producing them. More particularly, the present invention relates to compositions and methods for preparing bioenhanced benzoquinones utilizing at least one solubility-enhancing polymer. In accordance with certain embodiments, the benzoquinone is coenzyme Q10 (CoQ10); mixtures of benzoquinones are within the scope of the invention. In one embodiment, the mixture is prepared by dry blending the benzoquinone with a solubility-enhancing polymer. In another embodiment, the benzoquinone is dissolved in a solvent containing the polymer. In yet another embodiment, a blend of solvent/non-solvent for the polymer is employed. The bioenhanced benzoquinone product is produced by any method suitable to the composition. In one embodiment, direct compression of physically blended benzoquinone(sypolymer(s) is used. When necessary, solvent can be removed from compositions to yield the bioenhanced benzoquinone product. In one further development of the invention, CoQ10-polymer-solvent (or a solvent/non-solvent blend) is spray dried to produce CoQ10 in a form that exhibits improved solubility and/or bioavailability. The bioenhanced benzoquinone composition can be prepared by methods other than spray drying as recognized by those skilled in the art. Those methods include, without limitation: melt extrusion, spray congealing, and freeze drying. In accordance with particular embodiments of the invention, a significant portion of the benzoquinone is provided in the amorphous state. In accordance with certain embodiments, the benzoquinone is converted almost entirely to the amorphous state. In one preferred embodiment of the invention, the benzoquinone is converted to the completely amorphous state.

Coenzyme Q10(CoQ10, ubiquinone) is a lipid-soluble benzoquinone, a family of biochemicals produced either by aerobic organisms or through synthetic chemical processes. While a number of CoQ enzymes has been identified (e.g., CoQ6-Q10), only CoQ9 and CoQ10 are endogenous in man. Research suggests CoQ10 exerts powerful antioxidant and membrane stabilizing effects on the body, helps regulate metabolism, and may be important in patients with Alzheimer's, Parkinson's, and cardiac diseases especially coronary artery disease and congestive cardiac failure (Langade, 2005). Crystalline CoQ10 is essentially water-insoluble, which limits its bioavailability. Conventional dosage forms contain crystalline CoQ10 and, therefore, provide low bioavailability due to low CoQ10 aqueous solubility. As a result, conventional CoQ10 doses contain excessive amounts of CoQ10 in order to achieve a therapeutic effect. Other current commercial formulations typically present ubiquinone dissolved in soybean oil and glycerin (as a softgel) or as a dry powder capsule containing crystalline CoQ10. Alternatively, this compound has been reformulated with a variety of emulsifiers, lipids and oils in some soft gel products to enhance its bioavailability. However, soft gel technology is more labor- and cost-intensive process than capsule/tablet technologies. Furthermore, emulsified CoQ10 compositions are not well-suited for formulating with non-emulsified active ingredients.

It is desirable to produce solid compositions of benzoquinones and, in particular CoQ10, exhibiting enhanced solubility and/or bioavailability compared to the crystalline form of the compound. By converting a substantial portion of crystalline CoQ10 to the amorphous state, the aqueous solubility and bioavailability are increased. Furthermore, benzoquinones presented as an amorphous solid may facilitate manufacturing of the finished product and provide dosage forms that are substantially free of added lipids or oils or that may contain other active ingredients.

### SUMMARY OF THE INVENTION

The present invention provides compositions containing benzoquinone and methods for producing benzoquinone compositions, in particular CoQ10 compositions, of enhanced solubility and bioavailability. It has been discovered that mixtures of CoQ10 and solubility-enhancing polymers show enhanced aqueous solubility compared to crystalline CoQ10. Examples of compositions that create this enhancement include, without restriction: solid dispersions and physical blends of the components. Surprisingly, simple dry mixtures of CoQ10 and polymer attain dissolution release characteristics equal to many commercial softgel CoQ10 products, which employ lipids, oils and/or triglycerides. Even faster release with greater extent is produced with CoQ10-polymer dispersions, as shown in several embodiments of the invention. Although preferable, the amorphous conversion of CoQ10 is not a requirement for the enhanced properties.

A composition comprising a solid dispersion of a benzoquinone and at least one solubility-enhancing polymer wherein the benzoquinone in the dispersion is substantially amorphous is also provided. In one aspect, the disclosed invention describes the conversion of crystalline CoQ10 to the amorphous state. One method for producing this conversion is through solvent spray drying. Other techniques that accomplish this conversion include, without limitation: flash solvent evaporation, melt-congeal spraying, freeze drying, and melt-extrusion. These methods can use a single solubility-enhancing polymer or blends of polymers. Accordingly, products can be developed that serve the vegan/all natural market (e.g., using naturally-occurring ingredients/adjuvants) and a broader market (e.g., using synthetic ingredients/adjuvants). The degree of benzoquinone amorphous conversion depends on both polymer type and amount and processing conditions. When required, a single organic solvent, blends of solvents, or solvent/non- solvent blends can be used.

In one aspect, the invention relates to spray-dried powders or granulated products comprising amorphous benzoquinone. In addition, the resulting powders produced in accordance with certain embodiments typically possess lower residual solvent content and higher tap density than their counterparts produced by conventional methods, due to a change in the particle morphology and size.

One aspect of the invention involves amorphous benzoquinone prepared from compositions containing a benzoquinone and a solubility-enhancing polymer in a solvent or a solvent blend. This solvent or solvent blend includes a solvent in which the polymer is soluble. The term “soluble” means that the attractive force between polymer and solvent molecules is greater than the competing inter- and intramolecular attractive forces between polymer molecules. For simplicity, this solvent is simply called “solvent.” Compositions also are described in which the solvent blend contains a solvent for which the opposite is true: The force between polymer and solvent molecules is less than the inter- and intramolecular attractive force between polymer molecules. This second solvent is termed the “non-solvent.” The polymer may swell but does not dissolve in the non-solvent. In accordance with one embodiment of the invention, a solubility-enhancing polymer and a suitable solvent/non-solvent blend are provided. Additionally, the solvent possesses a lower boiling point than the non-solvent. Preferably, the solvent and non-solvent are miscible. The ratio of solvent to non-solvent is such that the polymer can be considered “dissolved” in the solvent system.

Unique particle properties can be created by evaporating the solvent/non-solvent blend. For example, this evaporation can occur during the spray drying of the feed solution or granulation processes. Atomized droplets containing a blend of solvents will experience a change in the total solvent composition due to evaporation. The method appears to be independent of how the droplets are generated or atomized. Initially, the polymer exists in a dissolved state, due to a sufficient amount of the solvent. As it evaporates (the solvent boils at a lower temperature than the non-solvent), the concentration of non-solvent in the droplet increases. Eventually, the solvent composition is insufficient to maintain the polymer in solution. In doing so, the polymer collapses from solution. This change in polymer conformation can alter the evaporation dynamics of the droplet to create particle morphologies that influence final powder properties.

Although benzoquinones of enhanced solubility and bioavailability can be formed by spray drying from a solution containing solvent alone, there are additional benefits associated with the use of a solvent/non-solvent blend system. This solvent/non-solvent approach can produce a spray dried powder of lower residual solvent content and smaller particle size. A further consequence of this engineered particle morphology is the increase in bulk powder density. Increased powder density is an important attribute for many applications. The extent of polymer collapse—and therefore the net effect on the spray dried powder properties—epends on the polymer solvation factors, such as the initial ratio of solvent to non-solvent, the polymer chemical structure and the polymer molecular weight. In addition to reducing residual solvent content and increasing density, the primary polymer may be paired with the solvent/non-solvent system in order to affect not only the morphology of the particle, but also that of the benzoquinone, and thereby affect active loading, crystallinity, solubility, stability and release.

The presence of additional polymers may contribute to the final particle morphology by their interaction with the first polymer and the solvent system. These additional polymers may also be advantageous to create special release properties of the active. For example, the primary polymer may be paired with the solvent/non-solvent system in order to affect particle morphology, and thereby residual solvent content and bulk powder density. Additional polymeric adjuvants may be added to serve additional purposes: further inhibit active recrystallization, further maximize active concentration, and further enhance/delay/retard dissolution rate. To accomplish these flnctionalities, it is necessary to suitably match the adjuvant solubilities with the solvent blend selected for the primary polymer.

### BRIEF DESCRIPTION OF THE DRAWINGS

Fig. 1A shows the dissolution profiles for the compositions of Example 1 (without added disintegrant) as described in Example 2;

Fig. 1B shows the dissolution profile for the completely amorphous composition of Example 1 (without added disintegrant) compared to two commercial CoQ10 products as described in Example 2.

FIG. 2A shows the dissolution profiles in USP water for a spray dried particle containing 1 CoQ10: 3 polyvinylpyrrolidone (without added disintegrant) compared to crystalline CoQ10;

FIG. 2B shows the dissolution profiles in USP water for a spray dried particle containing 1 CoQ 10: 3 polyvinylpyrrolidone (without added disintegrant) compared to two commercial CoQ10 products;

FIG. 3 shows the dissolution profiles for compositions of Example 1 (with added disintegrant) and for a commercial CoQ10 product as described in Example 4;

FIG. 4A shows the dissolution profiles for physical mixtures ofCoQ10 (without added disintegrant) and a commercial CoQ10 product in accordance with the experimental design of Example 5;

FIG. 4B shows the dissolution profiles for physical mixtures ofCoQ10 (with added disintegrant) and a commercial CoQ10 product in accordance with the experimental design of Example 5;

FIG. 5 shows the dissolution profiles in USP water for compositions of Example 6;

FIG. 6A is a photomicrograph of spray-dried 1 CoQ10: 3 polyvinylpyrrolidone particles from 100% solvent of Example 6;

FIG. 6B is a photomicrograph of spray-dried 1 CoQ10: 3 polyvinylpyrrolidone particles from solvent/non-solvent of Example 6;

FIG. 7 shows particle size distribution for CoQ10 spray dried dispersions from solvent and solvent/non-solvent approaches of Example 6;

FIG. 8 is a plot of heat flow versus temperature for crystalline CoQ10 and for CoQ10 spray dried particles from solvent and solvent/non-solvent approaches of Example 6;

FIG. 9 shows the dissolution profiles for one completely amorphous and one almost completely amorphous composition of Example 1 in pH 6.8 phosphate buffer as described in Example 7;

Fig. 10 shows the dissolution profiles for two tablet formulas containing spray dried 1 CoQ10 : 3 hydroxypropylmethyl cellulose phthalate in pH 6.8 phosphate buffer as described in Example 8;

FIG. 11 shows dissolution profiles for uncoated and coated tablets containing spray dried 1 CoQ10: 3 hydroxypropylmethyl cellulose phthalate in pH 6.8 phosphate buffer as described in Example 9;

FIG. 12 shows dissolution profiles for an uncoated tablet containing spray dried 1 CoQ10: 3 polyvinylpyrrolidone in pH 6.8 phosphate buffer as described in Example 10.

### DETAILED DESCRIPTION OF THE INVENTION

The term “comprising” encompasses the more restrictive terms “consisting essentially of'and “consisting of.”

All percentages, ratios and proportions used herein are by weight unless otherwise specified.

The term “solid dispersion” as used herein refers to a system in a solid state comprising at least two components, wherein one component is dispersed evenly throughout the other component or components. The term “solid dispersion” includes systems having small particles either completely crystalline, completely amorphous or any state in between, typically less than about 1 sum in diameter, of one phase dispersed in another phase.

The term “solid solution” as used herein refers to a type of solid dispersion wherein one component is molecularly dispersed throughout another component such that the system is chemically and physically uniform and homogeneous throughout. These systems do not contain any significant amounts of active ingredients in their crystalline or microcrystalline state as evidenced by thermal analysis (e.g., differential scanning calorimetry), or diffractive (e.g., X-ray diffraction) techniques.

There is no condition placed on the state of the compositions other than one or more benzoquinone(s) is combined with one or more solubility-enhancing polymer(s). The term “combined” includes, but is not limited to: blended, co-mingled, dissolved, extruded, granulated, melted, milled, mixed, sieved, slurried, sprayed, stirred, and the combination of these and other methods. Other techniques may be identified by those skilled in the art. Furthermore, compositions of the current invention may include additional active ingredients to the benzoquinone(s). Active pharmaceutical ingredients include, but are not limited to: analgesics, anti-arrhythmics, anti-bacterials, anti-convulsants, anti-Alzheimer's agents, anti-diabetics, anti-emetics, anti-fungals, anti-histiminics, anti-hyperlipidemics, anti-hyperlipoproteinemics, anti-hypertensives, anti-inflamatory agents, anti-Parkinsonian agents, anti-pulmonary hypertensives, anti-rheumatics, anti-ulceratives, anti-virals, cardiovascular agents, chemotherapy agents, central nervous system sedatives and stimulants, diuretics, gastrointestinal agents, hormones, respiratory agents, skin agents, as well as actives for the treatment of acne, benign prostatic hypertrophy, irritable bowel syndrome. Nutraceutical ingredients include, but are not limited to: herbs, isoflavones, moisturizers, mood regulators, minerals, oils, protein supplements, skin agents, ultraviolet blocking agents, and vitamins.

Although the following description is primarily directed to the preparation of a spray-dried composition containing CoQ10, the present invention is not limited to CoQ10 spray-dried compositions. The methods described herein are also useful in converting other benzoquinones to the amorphous state of enhanced solubility and bioavailability. Physical mixtures of benzoquinone and a solubility-enhancing polymer that increase the solubility and bioavailability of the benzoquinone are also within the scope of the present invention. Physical mixtures can be prepared in accordance with conventional techniques such as a tumble blender, high-shear granulation, fluid bed granulation, film coating, or any of their related technologies.

In accordance with one embodiment, the present invention is related to a method for preparing a spray-dried composition by providing a mixture containing CoQ10 and a polymer in a single solvent, a solvent blend or a blend of a solvent and a non-solvent for the polymer and spray drying the mixture to form the amorphous CoQ10 composition.

One aspect of the invention involves the pairing of the polymer with a carefully selected solvent or solvent blend. This approach comprises a solvent in which the polymer is soluble. Guidance in defining polymer solubility is provided by the expansion coefficient (α):

$$α = \frac{{( {\overline{r}}^{2} )}^{1 / 2}}{{( {\overline{{r}_{0}}}^{2} )}^{1 / 2}} \qquad ( §1 )$$

where r̅² is the mean-square distance between chain ends, and r̅ₒ̅² is the unperturbed dimension. (Equation § 1 can be written for branched polymers in an analogous manner, using square-average radius of gyration about the center of gravity, s̅² , and the corresponding unperturbed dimension, s̅ₒ̅².) Polymer solubility is provided when a is unity or greater, and solvents that satisfy this condition are called “good solvents,” or simply “solvents.” Solvents uncoil (or expand) the polymer molecule, since the polymer-solvent attractive force is greater than that of polymer-polymer. Light scattering methods, such as Viscotek's Triple Detector Array, can be used to determine the variables expressed in equation § 1. These concepts are defined in the text Polymer Chemistry, An Introduction, by Malcolm P. Stevens, which is incorporated by reference.

When a equals unity, a special condition exists in that polymer-solvent and polymer-polymer forces are balanced. Solvents that enable this condition are called θ solvents. Within the context of this invention, solvents are considered “good solvents” when α is about equal to 1 or more. It is appreciated that temperature influences α, such that a good solvent may be transformed into a non-solvent merely by changing the temperature.

In yet another embodiment of this invention, the solvent blend also contains a solvent for which the opposite is true: Polymer-polymer forces dominate polymer-solvent forces. In this case, a is less than one and the solvent is termed a “non-solvent,” because the polymer exists in a collapsed state. In accordance with one embodiment of the invention, the polymer is provided in a suitable solvent/non-solvent blend. The blend of solvent/non-solvent maintains a θ or solvated state of the polymer, such that the polymer can be considered “dissolved” in the solvent system. Additionally, the solvent possesses a lower boiling point than the non-solvent. (Solvent/non-solvent pairs that form an azeotrope do not satisfy this criterion.)

In accordance with another aspect of the invention, a polymer system is provided comprising a solubility-enhancing polymer and a suitable solvent/non-solvent blend. Specific examples of suitable polymer/solvent/non-solvent combinations include, without limitation, polyvinylpyrrolidone/dichloromethane/acetone, polyvinylpyrrolidone-co-vinyl acetate/acetone/hexane, and ethylcellulose/acetone/water. Unique particle architectures are created by precipitation of the primary polymer when the non-solvent concentration exceeds a critical value. This critical ratio R_{c} can be defined:

$${R}_{c} = \frac{mass nonsolvent}{mass solvent + nonsolvent} , \qquad ( §2 )$$

which is the maximum fraction of the non-solvent before polymer precipitation occurs. The ratio R_{c} for a given system can be determined experimentally by identifying the mass fractions of each component that produce a significant increase in solution turbidity. If an R_{c} value can be identified for a polymer system, then the system comprises a solvent/non-solvent blend. One example is a solution containing about 10% (w/w) polyvinylpyrrolidone, 18% (w/w) dichloromethane, and 72% (w/w) acetone, for which Rc equals 0.80. Polymer systems typically will be used at solvent/non-solvent blends that are at or below the R_{c} value for the system. It may be advantageous to formulate more complex polymer/solvent systems in order to control particle morphology/size as well as the crystallinity, solubility, bioavailability and release characteristics of the benzoquinone.

The present invention in accordance with other embodiments provides a method to increase the density of spray-dried powders. Typically, spray drying produces sphere-like particles with some degree of interior void. This void increases particle bulk without mass and creates low-density material. Adding a non-solvent to the working solution/dispersion changes the particle size and morphology, leading to an increase in density. Particles may be smaller, wrinkled, dimpled, and/or collapsed compared to those prepared using only solvent. The solvent/non-solvent approach also reduces the mean particle size, allowing the powder to pack better. In addition, powder flow and powder-powder mixing properties are enhanced.

The present invention in accordance with certain aspects provides a method to reduce or eliminate the need for secondary drying of spray-dried powders and granulated materials. These products often contain residual solvent, and it is desirable or necessary to produce a drier product. A high residual solvent content can result from formulation or processing limitations. The general practice has been to use a solvent that dissolves the solids being spray dried. In doing so, solvent can be trapped inside the spray dried powder or granulated bead due to case hardening. The intentional pairing of a lower-boiling solvent with a higher-boiling non-solvent for the materials being processed can yield products of lower residual solvent due to the effect(s) of the non-solvent on the process polymers.

The present invention may further provide a method to enhance the aqueous solubility and modify the release of active ingredients through selection of a polymer system with the solvent or solvent/non-solvent blend. The polymer system is chosen so that one (or more) polymer(s) work with the solvent/non-solvents to create novel particle morphologies. Additional polymer(s) may be added as needed to affect the solubility and release properties of the active, as well as particle morphology. Enhanced solubility can be achieved by a number of factors, including (but not limited to): improved wettability, creation of amorphous benzoquinone forms, stabilization against recrystallization, and/or co-solvation effects. In doing so, a supersaturatured solution of the benzoquinone is produced. “Modified release” refers to changing the time frame in which the active is released, i.e., immediate, delay, extended. These modified releases are created by matching functional polymer(s) with the appropriate solvent/non-solvent blend.

Solvents and non-solvents suitable for use in the process of the present invention can be any organic compound (including water) in which the primary polymer is soluble in the case of solvents, or insoluble, in the case of non-solvents. The choice and ratio of solvent/non-solvent depends on the choice of the primary polymer. Accordingly, the identification of an organic compound as a solvent or non-solvent depends on the primary polymer. Therefore, a solvent in one system may be a non-solvent in another. Particularly useful solvents and non-solvents include, but are not limited to: acetic acid, acetone, acetonitrile, anisole, 1-butanol, 2-butanol, butyl acetate, tert-butylmethyl ether, chlorobenzene, chloroform, cumene, cyclohexane, 1-2-dichloroethane, dichloromethane, 1-2-dimethoxyethane, N-N-dimethylacetamide, N-N-dimethylformamide, 1-4-dioxane, ethanol, 2-ethoxyethanol, ethyl acetate, ethylene glycol, ethyl ether, ethyl formate, formamide, formic acid, heptane, hexane, isobutyl acetate, isopropyl acetate, methanol, methyl acetate, 2-methoxyethanol, 3-methyl-i-butanol, methylbutylketone, methylcyclohexane, methylethyl ketone, methylisobutyl ketone, 2-methyl-i -propanol, N-methylpyrollidone, nitromethane, pentane, 1 -pentanol, 1 -propanol, 2-propanol, propyl acetate, pyridine, sulfolane, tetrahydrofuran, tetralin, 1-2-2-trichloroethene, toluene, water, and xylene. Mixtures of solvents and mixtures of non-solvents can also be used. In accordance with particular embodiments, solvent blends at the azeotropic composition (which boil at one common temperature) can comprise either the solvent or non-solvent, but not the solvent/non-solvent blend.

Solubility-enhancing polymers that are suitable for use in the mixtures of the present invention enhance the solubility of the benzoquinone. In accordance with particular aspects of the present inventions, the solubility-enhancing polymer also inhibits crystallization of the benzoquinone and, therefore, the presence of the polymer results in conversion of at least some of the crystalline benzoquinone to the amorphous state. In accordance with those embodiments wherein a solvent/non-solvent blend is used, at least one polymer should be soluble in the solvent and not soluble in the non-solvent. Specific examples of useful polymers include, but are not limited to: aliphatic polyesters (e.g., poly D-lactide), carbohydrates (e.g., sucrose), carboxyalkylcelluloses (e.g., carboxymethylcellulose), alkylcelluloses (e.g., ethylcellulose), gelatins, hydroxyalkylcelluloses (e.g., hydroxymethyl cellulose), hydroxyalkylalkyl celluloses (e.g., hydroxypropylmethyl cellulose), hydroxyalkylalkylcellulose derivatives, polyamines (e.g., chitosan), polyethylene glycols (e.g., PEG 8000, PEG 20000), methacrylic acid polymers and copolymers (e.g., Eudragite series of polymers of Rohm Pharma, GmbH), homo- and copolymers of N-vinyl pyrrolidone (e.g., polyvinylpyrrolidone, polyvinylpyrrolidone-co-vinyl acetate), homo- and copolymers of vinyllactam, polysaccharides (e.g., alginic acid), poly glycols (e.g., propylene glycol, polyethylene glycol), polyvinyl esters (e.g., polyvinyl acetate), and refined/modified shellac. The term “hydroxyalkylalkylcellulose derivatives” is meant to comprise hydroxypropylmethyl cellulose phthalate, and hydroxypropylmethyl cellulose acetate succinate. The amount of the polymer present in the mixture may range from about 1% to about 95%, more particularly from about 5% to 90%, by weight of the mixture, and in accordance with certain embodiments from about 25% to 75% by weight. Blends of polymers may also be used.

The bioenhanced composition, which may comprise a spray-dried mixture, includes a benzoquinone, such as CoQ10, as an active ingredient. The mixture may contain from about 1% to about 95% active, more particularly from about 20% to about 80% active, depending on the desired dose of the active. The weight ratio of benzoquinone to polymer typically will be from about 95% benzoquinone:5% total polymer to about 5% benzoquinone:95% total polymer, more particularly from about 70% benzoquinone:30% total polymer to about 30% benzoquinone:70% total polymer and in accordance with certain aspects from about 60% benzoquinone:40% total polymer to about 40% benzoquinone:60% total polymer.

The spray dried composition of the present invention when combined with a solubility enhancing polymer produces a portion of CoQ10 in the amorphous state. The term “amorphous” refers to a compound in a non-crystalline state. In other words, an amorphous compound lacks long-ranged, defined crystalline structure. In accordance with certain embodiments of the present invention, at least some, more particularly at least about 10%, at least about 25%, or at least about 40% of the benzoquinone in the composition is in an amorphous form. In other embodiments, at least a major portion of the compound in the composition is amorphous. As used herein, the term “a major portion” of the compound means that at least about 50% of the compound in the composition is in the amorphous form, rather than the crystalline form. More particularly, the compound in the composition may be substantially amorphous. As used herein, “substantially amorphous” means that the amount of the compound in the crystalline form does not exceed about 25% (i.e., more than about 75% of the compound is in the amorphous form). In accordance with particular embodiments of the invention, the compound in the composition is “almost completely amorphous” meaning that the amount of drug in the crystalline form does not exceed about 10% (i.e., more than about 90% of the compound is in the amorphous form). Compositions are also provided wherein the compound in the composition is considered to be “completely amorphous” meaning that the crystalline form of the drug is not detectable using conventional techniques, such as x-ray diffraction or thermal analysis. Reference to a composition as completely amorphous does not exclude compositions containing trace amounts (less than about 1%) of the crystalline form of the drug.

Amorphous materials lack some measurable properties, such as melting endotherms as measured by differential scanning calorimetry that characterize crystalline forms. Amounts of crystalline benzoquinone may be measured by powder X-ray diffraction (PXRD), differential scanning calorimetry (DSC), or any other standard quantitative analysis. The amounts of crystalline benzoquinone present in the composition may be detected by any other standard measurement known to those of ordinary skill in the art. It is appreciated that the measurement of such properties is dependent on instrument type, sensitivity, operation, and analysis.

By providing the CoQ10 in the amorphous form, the spray dried powder produced in accordance with certain aspects of the present invention provides enhanced solubility and/or bioavailability of CoQ10 compared to products containing the principle crystalline form (which melts at about 48° C.). The increased bioavailability of the active can also lead to reduced dosage sizes and dose amounts for the active. Applicants have also determined that the rate of CoQ10 release can be controlled through proper selection of the polymers added into the solvent solution for the spray dried process.

The spray dried mixture or bioenhanced composition may also contain additional polymeric materials that can modify properties of the composition. For example, certain polymers can be included to control particle morphology/size as well as the solubility and bioavailability and release characteristics of the active ingredient. Additional polymers may also be included in the mixture to further inhibit active recrystallization, further maximize active concentration and further enhance/delay/retard dissolution rate. Additional polymers that can be incorporated into this system are not particularly limited.

The mixture to be spray dried typically contains from about 40% to 99.9% by weight total solvent or solvent/non-solvent, more particularly from about 80% to 95% by weight total solvent or solvent/non-solvent based on the total weight of the mixture. When a solvent/non-solvent blend is used, the critical ratio R_{c} can vary from about 0.01-0.99, more particularly from about 0.1-0.9, still more particularly from about 0.3-0.8.

In addition to the solvent, polymer and CoQ10 or other benzoquinone, the mixture to be spray dried may also include other ingredients to improve performance, handling or processing of the mixture. Alternatively, these ingredients also may be admixed into the already-prepared benzoquinone-polymer by methods including, but not limited to tumble blending and granulation technologies. Typical ingredients include, but are not limited to, surfactants, pH modifiers, fillers, complexing agents, solubilizer, pigments, lubricants, glidants, flavor agents, plasticizers, taste masking agents, etc., which may be used for customary purposes and in typical amounts.

The spray drying apparatus used in accordance with certain aspects of the present invention can be any of the various commercially available apparatus or other devices capable of producing similar particles from liquid mixtures. Examples of specific spray drying devices include spray dryers manufactured by Niro Inc. (e.g., SD-Micro®, PSD-1®, PSD-2®, etc.), the Mini Spray Dryer®by Buchi Labortechnik AG, spray dryers manufactured by Spray Drying Systems, Inc. (e.g., models 30, 48, 72), and SSP Pvt. Ltd.

Spray drying processes and spray drying equipment are described generally in Perry's Chemical Engineers'Handbook, Sixth Edition (R. H. Perry, D. W. Green, J. O. Maloney, eds.) McGraw-Hill Book Co. 1984, pages 20-54 to 20-57. More details on spray drying processes and equipment are reviewed by Marshall “Atomization and Spray Drying,” 50 Chem. Eng. Prog. Monogr. Series 2 (1954). The relevant contents of these references are hereby incorporated by reference.

The term “spray drying” is used conventionally and, in general, refers to processes involving breaking up liquid mixtures into small droplets and rapidly removing solvent from the mixture in a container (spray drying apparatus) where there is a strong driving force for evaporation of solvent from the droplets. Atomization techniques include two-fluid and pressure nozzles, and rotary atomizers. The strong driving force for solvent evaporation is generally provided by maintaining the partial pressure of solvent in the spray drying apparatus well below the vapor pressure of the solvent at the temperatures of the drying droplets. This may be accomplished by either (1) maintaining the pressure in the spray drying apparatus at a partial vacuum; (2) mixing the liquid droplets with a warm drying gas; or (3) both.

Generally, the temperature and flow rate of the drying gas and the design of the spray dryer are chosen so that the polymer/active solution droplets are dry enough by the time they reach the wall of the apparatus that they are essentially solid and so that they form a fine powder and do not stick to the apparatus wall. It is also possible to operate a spray dryer so that product collects on the apparatus wall, and then is collected by removing the material manually, pneumatically, mechanically or other means. The actual length of time to achieve the preferred level of dryness depends on the size of the droplets, the formulation, and spray dryer operation. Following the solidification, the solid powder may stay in the spray drying chamber for 5-60 seconds, further evaporating solvent from the solid powder. The final solvent content of the solid dispersion as it exits the dryer should be low, since this improves the stability of the product. Generally, the residual solvent content of the spray-dried composition should be less than about 10% by weight and preferably less than about 2% by weight. In accordance with certain embodiments, the residual solvent content is within the limits set forth in the International Conference on Harmonization (ICH) Guidelines. Although not typically required in accordance with certain aspects of the present invention, because the presence of a non-solvent produces a spray-dried powder of lower residual solvent content, it may be useful in accordance with certain embodiments of the present invention to subject the spray-dried composition to further drying to lower the residual solvent to even lower levels. Methods to further lower solvent levels include, but are not limited to fluid bed drying, infra-red drying, tumble drying, vacuum drying, and combinations of these and other processes. Additional detail with respect to a particular spray drying process is described in more detail in the examples. However, the operating conditions to spray dry a powder are well known in the art and can be easily adjusted by the skilled artisan. Furthermore, the examples describe results obtained with a laboratory-scale spray dryer. One of ordinary skill in the art would readily appreciate variables that must be modified to obtain similar results with a production-scale unit.

As indicated above, the present invention is not limited to amorphous CoQ10 produced by spray drying. Applicants have determined that physical mixtures of CoQ10 with a solubility-enhancing polymer can also enhance the solubility and bioavailability of the CoQ 10. Methods for preparing physical mixtures of the polymer and CoQ10 are not particularly limited. In accordance with one aspect of the present invention, physical mixtures of solubility-enhancing polymer and CoQ10 may be formed by tumble blending, co-milling, stirring, granulating, or other methods known to those skilled in the art.

In addition to spray drying, compositions of the present invention may be prepared by other processes including, but not limited to, extrusion, spheronization and spray congealing.

Extrusion is a well-known method of applying pressure to a damp or melted composition until it flows through an orifice or a defined opening. The extrudable length varies with the physical characteristics of the material to be extruded, the method of extrusion, and the process of manipulation of the particles after extrusion. Various types of extrusion devices can be employed, such as screw, sieve and basket, roll, and ram extruders.

In melt extrusion, components can be melted and extruded with a continuous process with or without a solvent and with or without inclusion of other additives. Such a process is well-established and well-known to skilled practitioners in the art.

Spheronization is the process of converting material into spheres, the shape with the lowest surface area to volume ratio. Spheronization typically begins with damp extruded particles. The extruded particles are broken into uniform lengths instantaneously and gradually transformed into spherical shapes. In addition, powdered raw materials, which require addition of either liquid or material from a mixer, can be processed in an air-assisted spheronizer.

Spray congealing is a method that is generally used in changing the structure of the materials, to obtain free flowing powders from liquids and to provide pellets ranging in size from about 0.25 mm to 2.0 mm. Spray congealing involves allowing a substance of interest to melt, disperse, or dissolve in a hot melt of other additives. The molten mixture is then sprayed into an air chamber wherein the temperature is below the melting point of the formulation components, to provide spherical congealed pellets. The temperature of the cooled air used depends on the freezing point of the product. The particles are held together by solid bonds formed from the congealed melts. Due to the absence of solvent evaporation in most spray congealing processes, the particles are generally non porous and strong, and remain intact upon agitation. The characteristics of the final congealed product depend in part on the properties of the additives used. The feed rate and inlet/outlet temperatures are adjusted to ensure congealing of the atomized liquid droplet. The feed should have adequate viscosity to ensure homogeneity. The conversion of molten feed into powder is a single, continuous step. Proper atomization and a controlled cooling rate are critical to obtain high surface area, uniform and homogeneous congealed pellets. Adjustment of these parameters is readily achieved by one skilled in the art.

The spray congealing method is similar to spray drying, except that solvent is not used. Instead, the active ingredient(s) is dispersed and/or melted into a matrix comprising melt-processable polymer(s). Spray congealing is a uniform and rapid process, and is completed before the product comes in contact with any equipment surface. Most actives and additives that melt without decomposition are suitable for this method.

Conventional spray dryers operating with cool inlet air have been used for spray congealing. Several methods of atomization of molten mass can be employed, such as pressure, or pneumatic or centrifugal atomization. For persons skilled in the spray congealing art, it is well known that several formulation aspects, such as matrix materials, viscosity, and processing factors, such as temperature, atomization and cooling rate affect the quality (morphology, particle size distribution, polymorphism and dissolution characteristics) of spray congealed pellets. The spray congealed particles may be used in tablet granulation form, encapsulation form, or can be incorporated into a liquid suspension form.

Compositions prepared in accordance with certain aspects of the present invention provide amorphous benzoquinone that exhibits enhanced solubility and bioavailability without requiring the use of significant amounts of lipids or oils. In fact, certain aspects of the invention relate to compositions containing amorphous benzoquinone that are substantially free of lipids, triglycerides, or oils.

Benzoquinones produced in accordance with some embodiments of the invention exhibit enhanced solubility and bioavailability even when present in solid state forms such as solid solutions or solid dispersions. The benzoquinone may be present in such compositions at levels exceeding about 5% by weight, more particularly exceeding about 10%, and in some cases exceeding about 25%, 40% or even 50% by weight of the composition and still exhibit enhanced solubility and bioavailability compared to crystalline forms of the compound.

Compositions of the present invention may be delivered by a wide variety of routes, including, but not limited to: buccal, dermal, intravenous, nasal, oral, pulmonary, rectal, subcutaneous, sublingual, and vaginal. Generally, the oral route is preferred.

Compositions of the invention may be presented in numerous forms. Exemplary presentation forms are powders, granules, and multiparticulates. These forms may be added directly to capsules or may be further compressed to produce tablets, capsules, or pills, or reconstituted by addition of water or other liquids to form a paste, slurry, ointment, suspension or solution. Various additives may be mixed, ground, or granulated with the compositions of this invention to form a material suitable for the above dosage forms.

Compositions of the invention may be formulated in various forms so that they are delivered as a suspension of particles in a liquid vehicle. Such suspensions may be formulated as a liquid or as a paste at the time of manufacture, or they may be formulated as a dry powder with a liquid, typically water, added at a later time but prior to administration. Such powders that are constituted into a suspension are often referred to as sachets or oral powders for constitution (OPC). Such dosage forms can be formulated and reconstituted via any known procedure.

Oral, solid-dose spray dried powders typically have a mean particle size of about 0.5 μm-500 μm and are generally prepared from solutions at concentrations of 1% or more total solids, more particularly from about 2%-50%, and still more particularly from about 3%-30% solids.

Oral, solid dose granules typically have a mean particle size of about 50 μm-5000 μm. Techniques to produce granules include, but are not limited to, wet granulation and various fluid bed granulating methods.

Compositions comprising the benzoquinones of enhanced solubility and bioavailability described herein may be prepared in accordance with conventional techniques. In accordance with one aspect of the invention, a dosage form is provided comprising CoQ10 and a disintegrant. The disintegrant used in the composition is preferably of the so-called superdisintegrant type, disintegrants of this type being well-known to the person skilled in the art. As examples of these disintegrants the following can be mentioned: cross-linked polyvinylpyrrolidones, particularly crospovidone, modified starches, particularly sodium starch glycolate, modified celluloses, particularly croscarmellose sodium (cross-linked sodium carboxymethylcellulose) and LHPC (low-substituted hydroxypropyl cellulose). The disintegrant or superdisintegrant may be present in an amount of from about 2% to about 90%, preferably from about 3% to 60% of the composition.

The benzoquinone product produced by these compositions and methods described herein may be administered to man or animal. The compositions described herein may be administered as dietary supplements or as pharmaceutical compositions. The benzoquinone composition may be administered in a therapeutically effective amount to a human or animal in need of such treatment. The term “therapeutically effective amount” as used herein refers to an amount of a pharmaceutical ingredient that is effective to treat, prevent or alleviate the symptoms of a disease. The pharmaceutical compositions of the present invention may be used to treat a variety of diseases such as, but not limited to, Alzheimer's, Parkinson's, congestive heart failure and coronary artery disease. It can also be used as a nutrient, a nutritional supplement or a veterinary medicine.

The benzoquinone product described herein may be provided in various foods or beverages. Examples of suitable foods include baked goods and non-baked goods, such as nutritional bars, cakes, drink mixes and the like. Examples of beverages include waters, energy drinks, sport drinks, soft drinks, teas and the like.

The benzoquinone product described herein may also be provided in a semi-liquid (or semi-solid) form. Examples include, without limitation, ointments, creams, pastes, and salves. These compositions may be administered topically, orally, or sublingually.

Compositions prepared in accordance with certain aspects of the present invention preferably exhibit one or more of the following properties within the specified ranges:

| | CoQ10 COMPOSITION | | |
|---|---|---|---|
| PROPERTY | Broad | Intermediate | Narrow |
| release in aqueous media after 15 min | | | |
| absolute release | >5% | >20% | >80% |
| relative to crystalline CoQ10 | >10-times | >40-times | >160-times |
| release in aqueous media after 60 min | | | |
| absolute release | >10% | >40% | >80% |
| relative to crystalline CoQ10 | >20-times | >80-times | >160-times |
| degree of CoQ10 crystallinity | substantially | almost completely | completely |
| (Does not apply to physical mixtures.) | amorphous | amorphous | amorphous |
| | (<25% crystalline) | (<10% crystalline) | (<1% crystalline) |
| span of particle size distribution | ≦2 | ≦1.5 | ≦1 |
| tapped₁₂₅₀ density (g/mL) | >0.2 | >0.3 | >0.4 |

The present invention is described in more detail by the following non-limiting examples.

### Example 1

Four spray dried powders were made containing CoQ10 and two solubility-enhancing polymers at two CoQ10:polymer ratios for each polymer. The solutions for spray drying were prepared at 10% total solids by dissolving the polymer in solvent (dichloromethane for polyvinylpyrrolidone (PVP), acetone for hydroxypropylmethyl cellulose phthalate (HPMC-P) and then slowly adding CoQ10 until a solution was produced. Powders were produced using the SD-Micro® (Niro, Inc.) spray dryer with 0.5 mm ID, two-fluid nozzle. Analysis by modulated differential scanning calorimetry (MDSC) (Q1000®, TA Instruments) (0.5° C./min, heat-only conditions) showed the powders containing 75% polymer were completely or almost completely amorphous, while powders with 50% polymer contained some degree of crystalline CoQ10 (Table 1).

TABLE 1
Properties of spray dried CoQ10 with two solubility-enhancing
polymers at two polymer levels.

| | | PERCENT |
|---|---|---|
| | MASS RATIO | CRYSTALLINE |
| POLYMER | COQ10:POLYMER | COQ10 |
| polyvinylpyrrolidone | 1:1 | 18% |
| (Plasdone ® K-29/32, ISP) | 1:3 | 2% |
| hydroxypropylmethyl | 1:1 | 22% |
| cellulose phthalate | 1:3 | <1% |
| (HP-55, Shin Etsu) | | |

### Example 2

Dissolution properties were measured on the four spray dried powders of Example 1, crystalline CoQ10 and two commercial CoQ10 products (one soft gel and one dry powder capsule). All non-commercial samples were hand-filled into size 1 gelatin capsules (Shinogi Qualicaps). USP apparatus II (paddles) (VK 70100®, Varian Inc.) was used, with a bath temperature of 37° C. at 50 rpm for the first 60 minutes and then 200 rpm for an additional 15 minutes. The media contained Cremophoro EL (BASF Corp.), and 4% Acconono® MC8 (Abitec Corp.). Analysis was performed using high pressure liquid chromatography (HPLC) with UV detection (SCL-10 controller with SPD-10A detector module, Shimadzu Scientific Instruments)

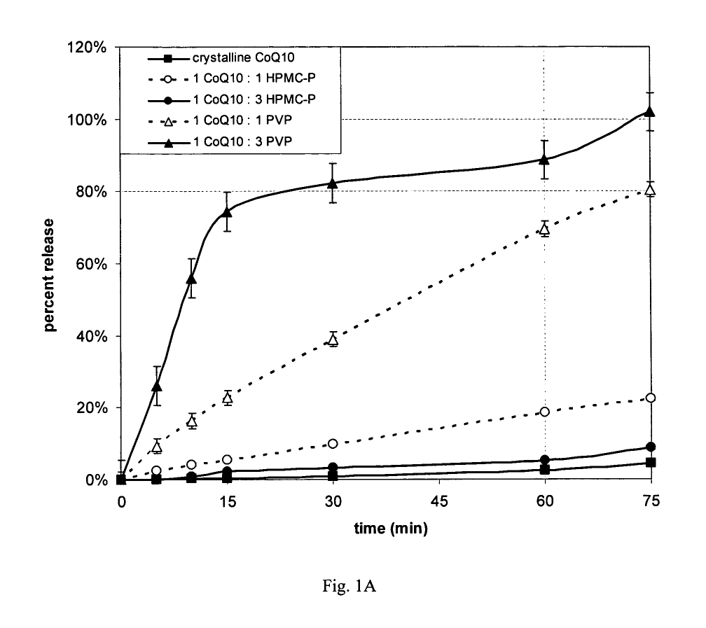

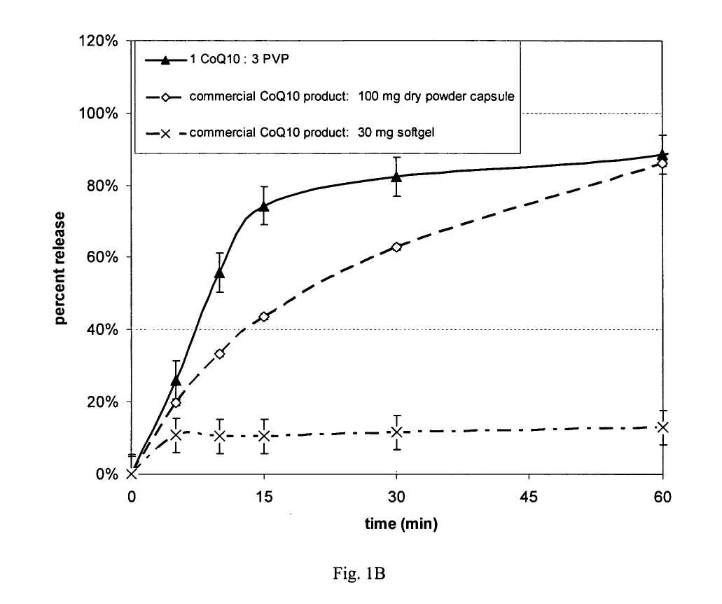

All spray dried CoQ10 products achieved faster dissolution with greater extent than pure ubiquinone (Fig. 1A). The rate and extent of release is controlled by the type and amount of polymer. In this dissolution media polyvinylpyrrolidone provided higher ubiquinone release than hydroxypropylmethyl cellulose phthalate. Increasing the polyvinylpyrrolidone content from 50% to 75% eliminated COQ10 crystallinity (Example 1) and further enhanced the rate of dissolution. The spray dried sample containing 1 part CoQ10 to 3 parts polyvinylpyrrolidone gave higher release than the commercially marketed CoQ10 products (Fig. 1B).

### Example 3

The dissolution behavior was measured in water without added surfactant for the completely amorphous spray dried particle from Example 1, crystalline CoQ10 and two commercial CoQ10 products. The dissolution test method remained identical as described in Example 2 except the dissolution medium contained only USP water.

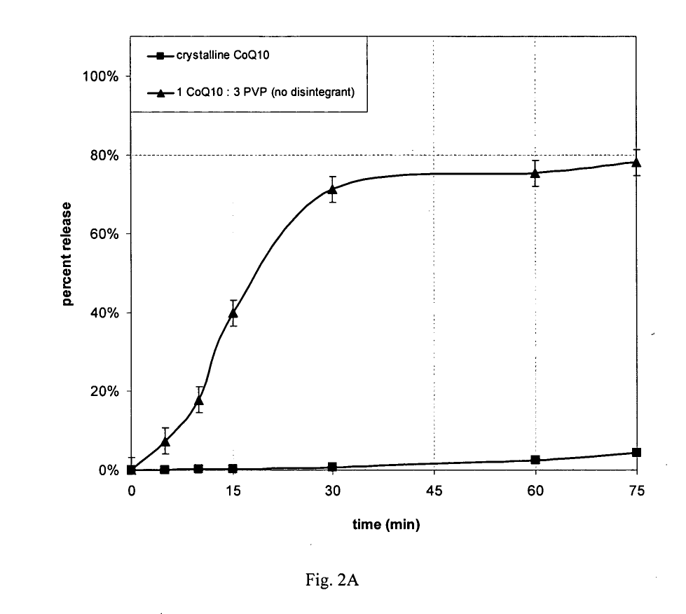

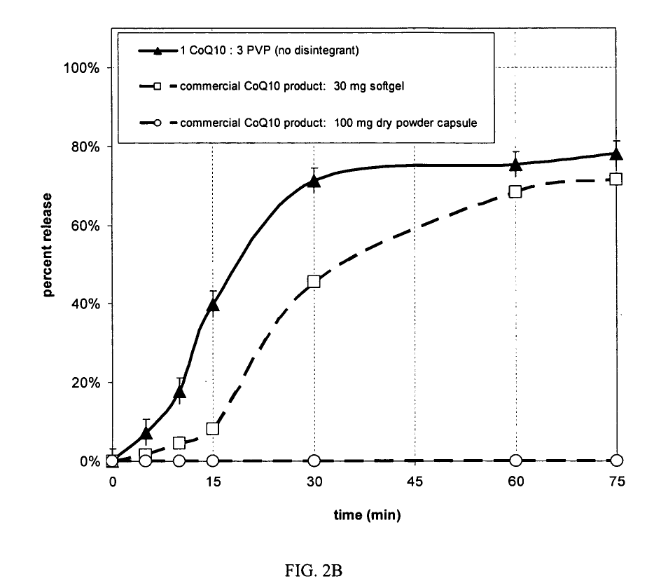

The completely amorphous 1 CoQ10: 3 polyvinylpyrrolidone product attained the fastest release with greatest extent of release relative to crystalline CoQ10 and the two commercial CoQ10 products (FIG. 2A and 2B). The rate of release after 10 minutes was 4.5-times higher for the capsule containing the amorphous spray dried powder (18% released) compared to the softgel capsule product (4% released). The commercial softgel product, which contained soybean oil, showed a lag in dissolution and lower maximum release, while the commercial product containing crystalline CoQ10 failed to give any release of the benzoquinone.

### Example 4

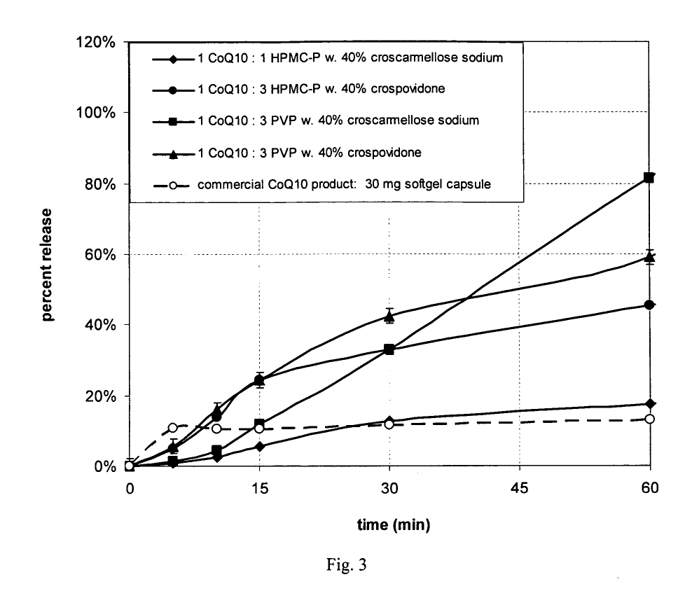

The samples produced in Example 1 were blended with two types of disintegrants, a small-particle size crospovidone (Polyplasdone® XL-10, ISP), and croscarmellose sodium of approximately the same size (Ac-Di-Solo®, FMC BioPolymer). The disintegrant level in each case was 40%. The dissolution procedure was the same as Example 2. The spray dried powders with disintegrant achieved comparable or higher CoQ10 release relative to a commerically market CoQ10 softgel product (FIG. 3).

### Example 5

Physical mixtures were prepared of crystalline CoQ10 with solubilizing polymers (PVP or HPMC-P). Powders were hand-filled into hard gelatin capsules (Shinogi Qualicaps) without and with an additional 40% disintegrant (small-particle crospovidone or croscarmellose sodium). Dissolution properties were measured using the method of Example 2.

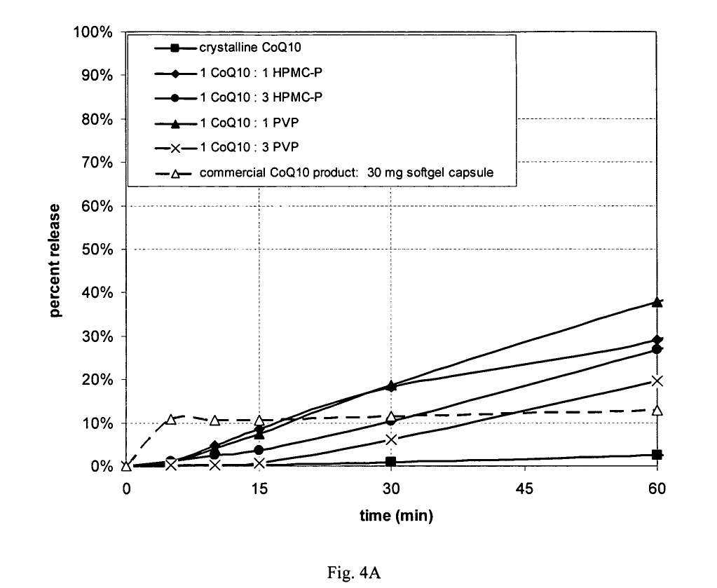

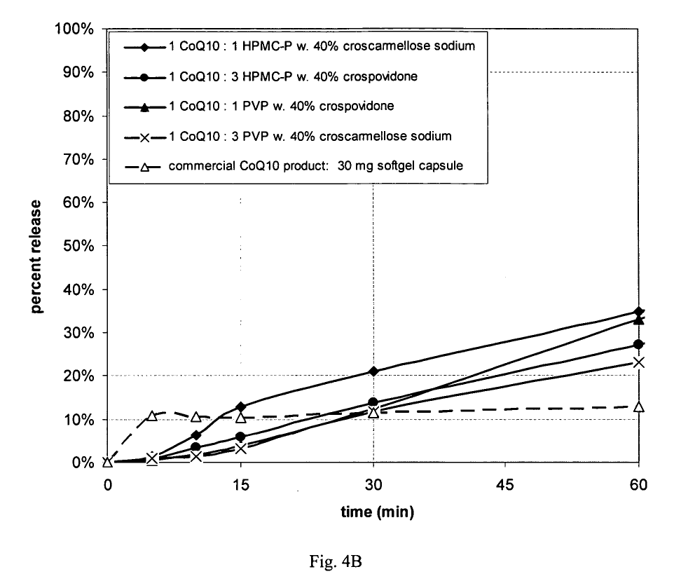

physical mixtures without disintegrant enhanced the release of CoQ10 compared to the crystalline form (FIG. 4A). The enhanced CoQ10 release ranged from 12-times higher (1 CoQ10: 3 PVP) to 28-times higher (1 CoQ10: 3 HPMC-P) compared to the crystalline form after 15 minutes. All physical mixtures without disintegrant provided higher CoQ10 release than the crystalline form after 60 minutes of dissolution testing.

All physical mixtures with disintegrant enhanced the release of CoQ10 compared to a commercial CoQ10 product (FIG. 4B).

It is surprising that simple physical mixtures of CoQ10 and solubilizing polymer (with or without added disintegrant) displayed enhanced dissolution behavior comparable to the crystalline CoQ10 form and a commercial CoQ10 product, which contains soybean oil but not solubilizing polymer. As one of ordinary skill in the art would appreciate the increase in solubility as evidence by the dissolution results is indicative of increased bioavailability of the active in the corresponding composition.

Example 6

CoQ10 was spray dried with polyvinylpyrrolidone (Plasdone® K-29/32, ISP) in the ratio 1 CoQ10: 3 polyvinylpyrrolidone at 20% total solids. Two spray dried powders were produced, one from 100% dichloromethane (DCM), the other from a blend of 80% DCM, 20% acetone. CoQ10 is soluble in both DCM and acetone, while polyvinylpyrrolidone is only soluble in DCM. All remaining spray drying conditions remained the same. Sample analysis included the dissolution method described in Example 2. Test results are provided in Table 2.

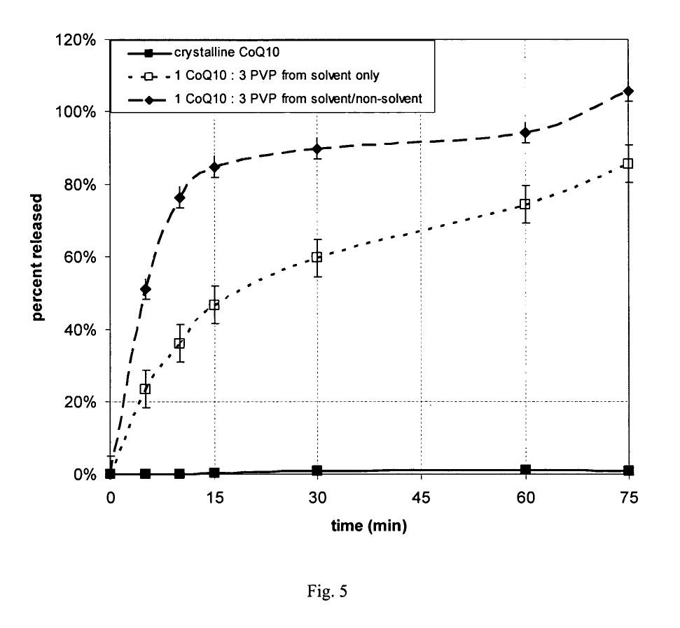

Both spray dried powders exhibited enhanced dissolution properties compared to crystalline CoQ10 (FIG. 5). The change in solvent system affected dissolution behavior, in that powder from solvent/non-solvent solution attained remarkably faster and higher release and extent of release. It is surprising that the time for 50% CoQ10 dissolution (t₅₀%) was shortened 260%, from 18 minutes to 5 minutes by the switch from 100% solvent to the solvent/non-solvent blend. Similarly, the time for 80% CoQ10 dissolutin (t₈₀%) was shortened from 68 minutes to 12 minutes by the switch from 100% solvent to the solvent/non-solvent blend.

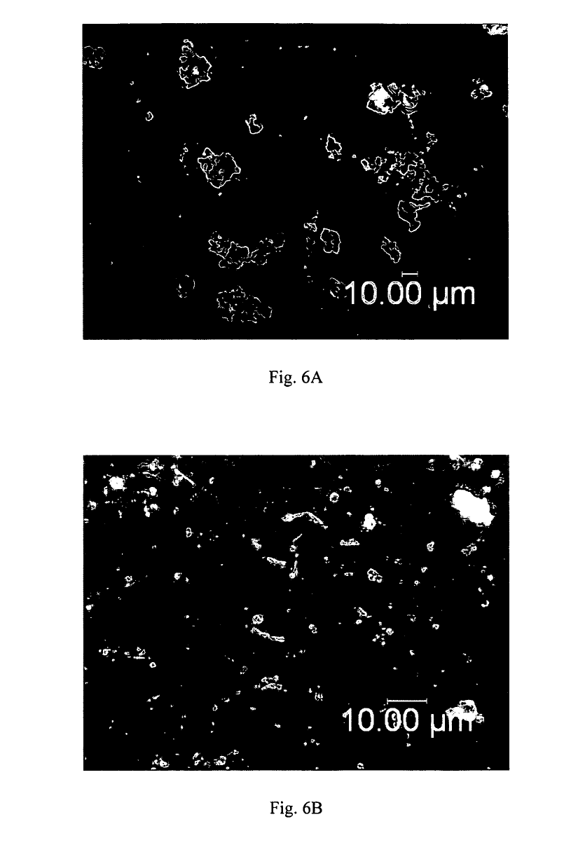

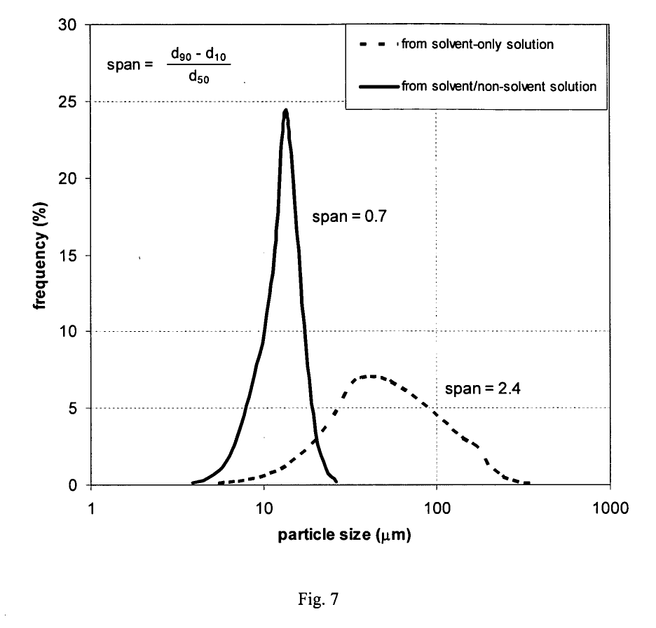

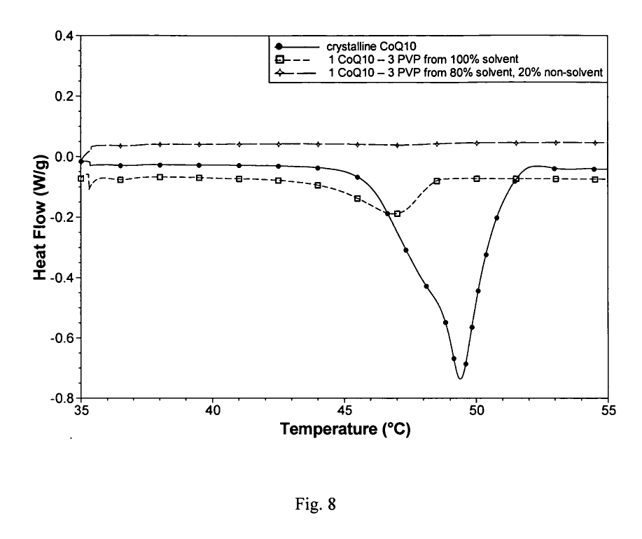

Particles from solvent solution were spherical/globular (FIG. 6A), while smaller, thread-like particles formed from the solvent/non-solvent blend (FIG. 6B). The change from solvent to solvent/non-solvent also reduced the particle size distribution (FIG. 7), as indicated by a reduced span, as measured by a laser scatting method in air (LA-910®, Horiba Instruments) (Table 2). The span was reduced 71%, from 2.4 (solvent-only method) to 0.7 (solvent/non-solvent method).

Surprisingly, the change in solvent system also altered the CoQ10 physical chemistry. While both products were less crystalline than the starting material, the sample prepared from the solvent/non-solvent blend was almost completely amorphous, while significant crystallinity was measured in the spray dried powder from 100% DCM under these conditions (FIG. 8).

Other advantages of the solvent/non-solvent blend approach include lower residual solvent content, as measured by a moisture balance (MB45, Ohaus Corp.) and higher density, as measured by a tap densitometer (TD- 1020, Distek Inc.)

TABLE 2
Characteristics of CoQ10 sprayed dried from
solvent and solvent/non-solvent solutions.

| | | spray dried dispersion | |
|---|---|---|---|
| | CoQ10 | | from solvent/ |
| property | (as received) | from solvent | non-solvent |
| crystallinity* | 100% | 72% | 3% |
| max. CoQ10 release | 4.3% | 85.8% | 100% |
| t_{50%} | not attained | 18 min | 5 min |
| | within 75 minutes | | |
| t_{80%} | not attained | 68 min | 12 min |
| | within 75 minutes | | |
| spray dry yield | | 63% | 83% |
| loss on drying | | 3.8% | 2.5% |
| particle morphology | | spherical, | fine fragmented |
| | | globular | threads and |
| | | | microspheres |
| median particle size: | | 45 μm | 12 μm |
| d₅₀ (μm) | | | |
| particle size | | 2.4 | 0.7 |
| distribution: span^{†} | | | |
| bulk density | | 0.132 | 0.154 |
| (g/mL) | | | |
| tapped₁₂₅₀ density | | 0.179 | 0.216 |
| (g/mL) | | | |

*As measured by MDSC using heat-only conditions with aluminum,
hermetic pans.
${}^{†} As measured by laser scattering method in air , span = \frac{{d}_{90} - {d}_{10}}{{d}_{50}} .$

### Example 7

Dissolution properties were measured for two of the amorphous spray dried powders of Example 1 and compared to crystalline CoQ10. The first contained 1 CoQ10 : 3 HPMC-P and the second contained 1 CoQ10: 3 PVP. The tested dose of CoQ10 was 30 mg, and the materials were filled into hard gelatin capsules (Shinogi Qualicaps) with additional 15% croscarmellose sodium (Ac-Di-Sole, FMC BioPolymer). The dissolution test conditions were identical to those of Example 2, except the dissolution medium was pH 6.8 phosphate buffer. The solubility of hydroxypropylmethylcellulose phthalate is pH dependent, dissolving in gastrointestinal fluids with a pH greater than about 5. The solubility of polyvinylpyrrolidone is pH-independent.

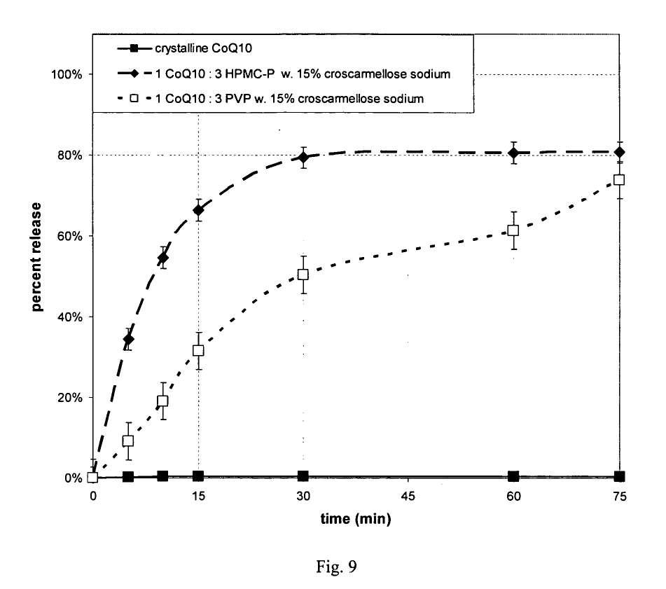

Both amorphous spray dried dispersions provided higher and faster release than the crystalline form (FIG. 9). Compared to the crystalline form after 15 minutes, the PVP formulation enhanced CoQ10 release 88-fold, while the HPMC-P formula enhanced CoQ10 release 189-fold. Even greater enhanced release was measured after 60 minutes of testing: 192-fold (HPMC-P formula vs. crystalline), and 174-fold (PVP formula vs. crystalline).

### Example 8

The amorphous spray dried powder of Example 1 containing 1 CoQ 10: 3 HPMC-P was tableted using two formulations. The tablet dissolution properties were measured in pH 6.8 phosphate buffer using the same method as Example 7. These tablet formulations were otherwise identical except for the choice of diluent: The first contained microcrystalline cellulose (Avicel® PH102, FMC BioPolymer), while the second contained lactose monohydrate (Fast-Flo®316, Foremost Ingredients Group) (Table 3).

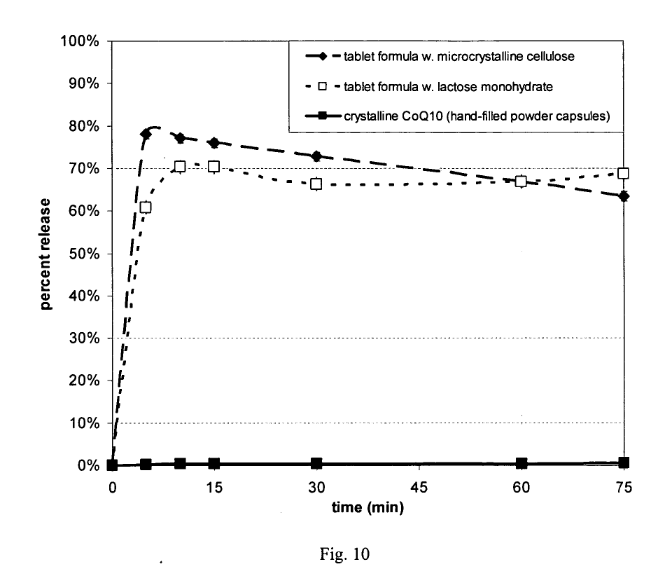

Both uncoated tablet formulations maintained high release of CoQ10 in pH 6.8 phosphate buffer, increasing CoQ10 release by about 200-times compared to crystalline CoQ10 (FIG. 10).

TABLE 3
Tablet formulas for prototypes of Example 8.

TABLET FORMULA

| INGREDIENT | 8A | 8B |
|---|---|---|
| 1 CoQ10:3HP-55 | 30% | 30% |
| amorphous spray dried dispersion | | |
| microcrystalline cellulose | 59% | |
| (Avicel ® PH102) | | |
| anhydrous lactose | | 59% |
| (Fast-Flo ® 316) | | |
| croscarmellose sodium | 10% | 10% |
| (Ac-Di-Sol) | | |
| silica | 0.5% | 0.5% |
| (Cabosil M5P) | | |
| magnesium stearate | 0.5% | 0.5% |

### Example 9

Tablets made by formula 8A of Example 8 were coated to 3% weight gain (dry weight basis) using a coating comprising about 32% hydroxypropylmethylcellulose (6 cP), about 5% glycerin, and about 63% aqueous shellac. Tablet dissolution was measured in pH 6.8 using the same method as described in Example 7.

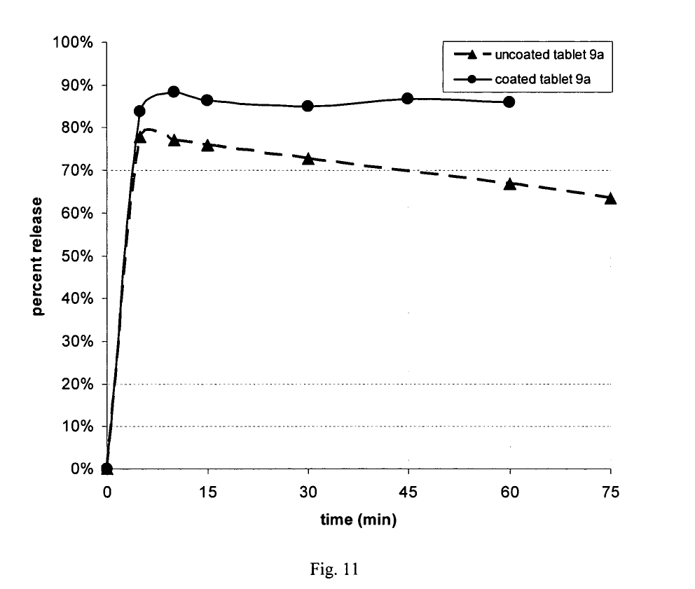

It is surprising that the coated tablet achieved yet higher and more constant CoQ10 release than the uncoated tablet (FIG. 11).

### Example 10

The amorphous spray dried powder of Example 1 containing 1 CoQ 10: 3 polyvinylpyrrolidone (Plasdone® K-29/32, International Specialty Products) was tableted using standard tabletting excipients (Table 4). Tablets contained 30 mg CoQ10. Dissolution properties were measured in pH 6.8 phosphate buffer using the method of Example 7.

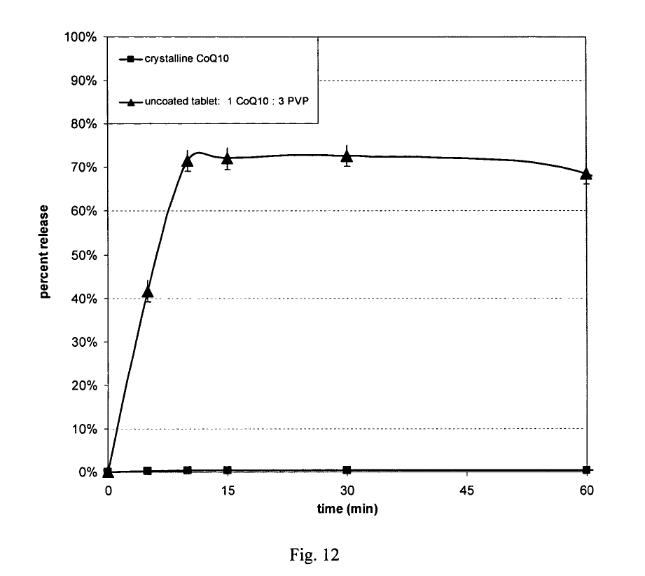

After 15 minutes of dissolution, the PVP-containing tablet enhanced CoQ10 release by 200-times compared to crystalline CoQ10 (FIG. 12).

TABLE 4
Tablet formulation for amorphous spray
dried powder containing 1 CoQ10:3 PVP.

| INGREDIENT | WEIGHT PERCENT |
|---|---|
| 1 CoQ10:3 PVP amorphous | 30% |
| spray dried powder | |
| anhydrous lactose | 59% |
| (Fast-Flo ® 316) | |
| croscarmellose sodium | 10% |
| (Ac-Di-Sol ®) | |
| silica | 0.5% |
| (Cabosil ® M5P, Cabot Corp.) | |
| magnesium stearate | 0.5% |

### Example 11

The single-dose pharmacokinetics of CoQ10 compositions disclosed in this invention are compared to a crystalline CoQ10 product in healthy human males in the fasted state. The uncoated tablets of Example 8 (tablet 8A) and Example 10 and a commercial CoQ10 product containing 100 mg crystalline CoQ10 in a dry powder, hard gelatin capsule are selected. Human volunteers are single-dosed at 300 mg after 8 hours without food. Blood samples are drawn and a validated analytical method is used to measure the blood plasma concentration of CoQ10 as a ftmction of time after dosing.

CoQ10 tablets of this invention are expected to achieve higher blood plasma concentrations of CoQ10 relative to the crystalline, commercial product.

Changes may be made by persons skilled in the art in the compositions and/or in the steps or the sequence of steps of the method of manufacture described herein without departing from the spirit and scope of the invention as defined in the following claims.

What is claimed is:

1. A composition comprising a benzoquinone and a solubility-enhancing polymer wherein said benzoquinone exhibits enhanced bioavailability compared to a control composition without the solubility-enhancing polymer.

2. The composition of claim 1 wherein said composition comprises a physical mixture of benzoquinone with the polymer(s).

3. The composition of claim 1 wherein the benzoquinone comprises CoQ10.

4. The composition of claim 1 wherein the polymer is selected from the group consisting of: aliphatic polyesters, carbohydrates, carboxyalkyl celluloses, alkyl celluloses, hydroxyalkyl celluloses, hydroxyalkylalkyl celluloses, hydroxyalkylalkyl cellulose derivatives, polyamines, polyethylene glycols, methacrylic acid polymers and copolymers, homo- and copolymers of N-vinyl pyrrolidone, homo- and copolymers of vinyllactam, polysaccharides, poly glycols, polyvinyl esters, refined/modified shellac, and mixtures thereof.

5. The composition of claim 4 wherein the polymer comprises a hydroxyalkylalkyl cellulose derivative selected from the group consisting of hydroxypropylmethyl cellulose acetate succinate (HPMCAS), hydroxypropylmethyl cellulose phthalate and combinations thereof.

6. The composition of claim 1 wherein the ratio of benzoquinone to total polymer is between about 5% benzoquinone:95% total polymer to about 95% benzoquinone:5% total polymer.

7. The composition of claim 6 wherein the polymer comprises polyvinylpyrrolidone or polyvinylpyrrolidone-co-vinyl acetate.

8. The composition of claim 2 wherein the physical mixture comprises a simple blend of the components or a granulation.

9. The composition of claim 1 wherein the composition comprises spray dried particles wherein the particles comprise benzoquinones and the solubility-enhancing polymer.

10. A composition in accordance with claim 9 wherein the composition is provided in the form of an oral, solid-dosage form.

11. A composition in accordance with claim 10 wherein the oral, solid-dosage form comprises a tablet, a coated tablet, a chewable tablet, a capsule or a gelatin capsule.

12. A composition in accordance with claim 9 wherein the composition is in the form of a paste, solution, slurry, ointment, or dispersion.

13. The composition of claim 1 further comprising one or more ingredients selected from the group consisting of surfactant(s), pH modifier(s), filler(s), complexing agent(s), solubilizer(s), pigment(s), lubricant(s), glidant(s), flavor agent(s), plasticizer(s), taste masking agent(s), release-modifying polymer(s), and mixtures thereof.

14. A composition in accordance with claim 1 wherein the composition is in the form of an oral, solid-dosage form.

15. A composition in accordance with claim 14 wherein the oral, solid-dosage is selected from the group consisting of a tablet and a capsule.

16. A composition in accordance with claim 15 wherein the oral, solid-dosage form comprises a coated tablet, chewable tablet, or gelatin capsule.

17. A personal care product or cosmetic product compromising the composition of claim 1.

18. A food or beverage product compromising the composition of claim 1.

19. A composition in accordance with claim 1 wherein the composition is in the form of a paste, solution, slurry, ointment, or dispersion.

20. The composition of claim 1 wherein the composition comprises amorphous benzoquinones.

21. The composition of claim 1 wherein the composition is melt-processable.

22. A composition in accordance with claim 21 wherein the composition is provided in the form of an oral, solid-dosage form.

23. A composition in accordance with claim 22 wherein the oral, solid-dosage form comprises a tablet, a coated tablet, a chewable tablet, a capsule or a gelatin capsule.

24. A composition in accordance with claim 21 wherein the composition is in the form of a paste, solution, slurry, ointment, or dispersion.

25. A method of preparing a composition comprising a benzoquinone comprising:
contacting a benzoquinone with a solubility-enhancing polymer in a solvent for the polymer, thereby forming a mixture containing a benzoquinone of enhanced bioavailability.

26. The method of claim 25 further comprising removing the solvent to form a benzoquinone-polymer composition.

27. The method of claim 25 wherein the benzoquinone comprises CoQ 10.

28. The method of claim 25 wherein the ratio of benzoquinone to total polymer is between about 5% benzoquinone:95% total polymer to about 95% benzoquinone:5% total polymer.

29. The method of claim 25 wherein the composition further comprises one or more pharmaceutically acceptable ingredients.

30. The method of claim 25 wherein the polymer is selected from the group consisting of: aliphatic polyesters, carboxyalkyl celluloses, carbohydrates, alkyl celluloses, hydroxyalkyl celluloses, hydroxyalkylalkyl celluloses, hydroxyalkylalkyl cellulose derivatives, polyamines, polyethylene glycols, methacrylic acid polymers and copolymers, homo- and copolymers of N-vinyl pyrrolidone, homo- and copolymers of vinyllactam, polysaccharides, poly glycols, polyvinyl esters, refined/modified shellac, and mixtures thereof.

31. The method of claim 30 wherein the polymer is selected from the group consisting of polyvinylpyrrolidone, hydroxypropylmethylcellulose phthalate, polyvinylpyrrolidone-co-vinyl acetate and HPMCAS.

32. The method of claim 31 wherein the polymer comprises polyvinylpyrrolidone.

33. The method of claim 31 wherein the polymer comprises hydroxypropylmethylcellulose phthalate or HPMCAS.

34. The method of claim 31 wherein the polymer comprises cross-linked polyvinylpyrrolidone (crospovidone).

35. The method of claim 25 wherein the mixture further comprises a non-solvent for the polymer.

36. The method of claim 35 wherein the solvent and non-solvent are present at a ratio of from about 5% solvent: 95% non-solvent to about 95% solvent: 5% non-solvent.

37. The method of claim 36 wherein the ratio of solvent to non-solvent is selected such that the polymer is dissolved in the solvent blend.

38. The method of claim 35 wherein the benzoquinone of enhanced bioavailability exhibits faster dissolution, greater extent of dissolution, or both compared to a benzoquinone composition made without a non-solvent for the polymer.

39. The method of claim 38 wherein the benzoquinone of enhanced bioavailability exhibits a reduction in the time for 50% benzoquinone dissolution by about 100% or more compared to a benzoquinone composition made without a non-solvent for the polymer.

40. The method of claim 25 wherein the concentration of the polymer in the mixture is from about 1% to about 90%.

41. The method of claim 26 wherein the solvent is removed by spray drying the mixture to form particles comprising benzoquinone.

42. The method of claim 41 wherein said particles contain less than about 2% residual solvent.

43. The method of claim 25 wherein a major portion of said benzoquinone in said mixture is amorphous.

44. The method of claim 43 wherein the benzoquinone in said mixture is almost completely amorphous.

45. A composition comprising particles produced in accordance with claim 41.

46. The method of claim 35 wherein the benzoquinone product produced from the solvent/non-solvent method reduces the span of the particle size distribution by about 50% or more compared the benzoquinone product produced from solvent-only solution

47. An oral, solid-dosage form comprising particles produced in accordance with claim 41.

48. The oral, solid dosage form of claim 47 in the form of a capsule, a tablet, a chewable tablet, a granule, a bead, a gelatin capsule, or a pellet.

49. A composition in accordance with claim 1 further comprising at least one other active ingredient.

50. A composition in accordance with claim 49 wherein the other active ingredient is a nutraceutical ingredient.

51. A composition in accordance with claim 47 wherein the other active ingredient is an active pharmaceutical ingredient.

52. A composition comprising a benzoquinone produced in accordance with claim 25.

53. A method for providing benzoquinone to a subject comprising administering to said subject the oral, solid dosage form of claim 47.

54. The method of claim 53 wherein said dosage form comprises a nutraceutical product.

55. The method of claim 53 wherein said dosage form comprises a pharmaceutical product.

56. The method of claim 53 wherein said dosage form is administered to treat congestive heart failure.

57. The method of claim 53 wherein said dosage form is administered to treat coronary artery disease.

58. A pharmaceutical composition comprising a benzoquinone and at least one pharmaceutically acceptable excipient wherein the benzoquinone comprises spray dried benzoquinone in an amorphous state.

59. The composition of claim 58 wherein said pharmaceutical composition is substantially free of lipids or oils.

60. A composition comprising a solid dispersion of a benzoquinone and at least one solubility-enhancing polymer wherein said benzoquinone in said dispersion is substantially amorphous.

61. The composition of claim 60 wherein said benzoquinone in said dispersion is almost completely amorphous.

62. The composition of claim 60 wherein said benzoquinone in said dispersion is completely amorphous.

63. The composition of claim 60 wherein at least about 20% of the benzoquinone is released in 15 minutes when tested in aqueous media.

64. The composition of claim 63 wherein the benzoquinone is in the form of spray dried particles.

65. The composition of claim 64 wherein the span of the particle size of the spray dried particles is less than about 1.5.
